(19) United States
(12) Patent Application Publication
(10) Pub. No.: US 2009/0270910 A1
(43) Pub. Date: Oct. 29, 2009

(54) Method and Apparatus for Increasing Blood Flow in a Body Part

(75) Inventors: Alan R. Hargens, San Diego, CA (US); Brandon R. Macias, College Station, TX (US); Timothy B. Neuschwander, La Jolla, CA (US); Qiuxia Zhang, Goteborg (SE)

Correspondence Address: PROCOPIO, CORY, HARGREAVES & SAVITCH LLP, 530 B STREET, SUITE 2100, SAN DIEGO, CA, 92101, US

(73) Assignee: THE REGENTS OF THE UNIVERSITY OF CALIFORNIA, Oakland, CA (US)

(21) Appl. No.: 12/301,017

(22) PCT Filed: May 18, 2007
(86) PCT No.: PCT/US07/69314

§ 371 Date: Nov. 14, 2008

Related U.S. Application Data
(60) Provisional application No. 60/802,215, filed on May 19, 2006.

(51) Int. Cl. A61B 17/00

(52) U.S. Cl. 606/201

(57) ABSTRACT

An enclosure is dimensioned to enclose an affected area of a body part plus a small margin around which a releasable seal is attached to make the enclosure substantially airtight around the body part without contacting the area to be treated. A port in the enclosure is attached to tubing for connection of a pump for forcing air under pressure into the enclosure. In the preferred embodiment, the enclosure is an inflatable plastic bag. The compression provided by the air pressure is applied uniformly to the body part to increase blood flow within the body part without physically contacting the affected area of the body part. In one embodiment, the pump is a combination air and vacuum pump so that pressures within the enclosure may be cycled between high and low pressures.

### RELATED APPLICATIONS

This application claims priority of U.S. Provisional Application No. 60/802,215, filed May 19, 2006, which is incorporated herein by reference in its entirety.

### FIELD OF THE INVENTION

The present invention relates to a method and apparatus for increasing blood flow to a body part, and more specifically to a method and apparatus that uses air pressure rather than physical contact with the area that is being treated.

### BACKGROUND OF THE INVENTION

Patients suffering from an injury or illness that impairs blood circulation in a limb or other body part require enhancement of the circulation in order to heal, or in extreme cases, to save the limb and provide for full recovery. Inadequate blood flow into and out of the injured limb can lead to such problems as pain upon exertion of the limb, slow healing of injuries, breakdown of soft and hard tissues leading to slow healing of tissues or even gangrene, which can lead to amputation of the affected limb.

A major source of morbidity for patients with diabetes mellitus is foot ulcers. It has been estimated that foot ulcers occur in 2.5% of diabetic patients each year. Moreover, diabetes is also the main cause of non-traumatic lower extremity amputations in orthopedics. Surgical revascularization sometimes cannot be performed for these patients due to poor peripheral circulation. Conservative treatments involving the use of dressings and other wound-care products are only adjuncts to careful local treatment, including pressure reduction for foot (crutch, wheelchair, walker), wound debridement, and infection control. Use of vasodilator drugs does not aid in the healing of diabetic foot ulcers. Hyperbaric oxygen is occasionally effective, however raising the oxygen content of the blood is of less value when the blood supply to the foot is severely impaired.

A number of prior art techniques have involved local application of mild compression to the affected area to increase blood flow. Compression therapies may contribute to increase blood flow in the limb by one or more of the following mechanisms: veno-arteriolar response, venous pumping, and a myogenic response. Compression therapies have been reported to increase skin and muscle blood flow, decrease venous stasis ulcer healing times, increase capillarity, and even decrease healing time of fractures.

Existing applications of compression therapy can be divided into two main categories: intermittent pneumatic compression and continuous compression. Intermittent pneumatic compression devices usually consist of a device that expands an annular air bladder surrounding the body part for a specified period of time, followed by a period of time of deflation. These devices may operate within ranges around 40 mmHg up to 120 mmHg above ambient pressure (˜760 mmHg). They are generally marketed for applications of wound healing, preventing thrombosis and reducing edema in the extremities. Continuous compression devices usually consist of a garment with graded compression, and operate in the range of 15 to 50 mmHg.

Existing devices exhibit a number of disadvantages, including that they all directly contact the body part in some manner to apply compression (via an inflatable air bladder or elastic compression garment). By touching the skin, existing devices can be problematic for post-surgical patients or other patients in whom direct cutaneous compression may cause pain, compression of delicate surgical repairs, or contamination of the wound site. Further, they will not work on patients with casts or external fixators since they operate by physically applying pressure against the skin, or against a sterile fabric directly covering the skin. When compression is employed on a post-surgical patient, a multi-layer compression bandage system is frequently used. This makes wound inspection difficult, since the bandage must be removed and replaced every time a wound check is performed.

Tests have shown that prior art compression devices that physically contact the surface do not uniformly compress the body part. For example, one of the present inventors reported on pressure isobars under tourniquets, showing that tourniquet pressures decrease by approximately 50% from the skin to the bone. (Hargens A. R., et al., “Local compression patterns beneath pneumatic tourniquets applied to arms and thighs of human cadavera,” Journal of Orthopaedic Research 5:247-252, 1987). Since intermittent pneumatic compression devices operate in much the same manner as the pneumatic tourniquets, it is expected that they similarly would not provide uniform compression. Some reports have indicated that intermittent pneumatic compression with several-second pulses of pressure followed by 10-30 seconds of rest provide only a limited increase in popliteal artery peak systolic velocity, typically by only about 20-25%.

Accordingly, the need remains for a device and method for effectively enhancing blood flow in a body part, from skin to bone, without subjecting the body part to conditions that counteract the beneficial effects of the treatment, such as directly contacting wounds. The present invention is directed to such a need.

### BRIEF SUMMARY OF THE INVENTION

It is an advantage of the present invention to provide a device and method for applying uniform compression to a body part for enhancing microvascular flow in skin, muscle and bone without contacting the area under treatment.

Another advantage of the present invention is to provide a device for enhancing microvascular flow in a body part that is easily applied and removed to permit access for inspection of the body part.

Still another advantage of the present invention is to provide a device and method for increasing popliteal artery inflow to the body part.

According to the present invention, blood flow to the target body part, e.g., a leg, arm or a portion of the torso, is increased by applying external air pressure around the body part, without physically contacting the affected area with any part of the device.

In an exemplary embodiment, the inventive device comprises a substantially air-tight enclosure that is dimensioned to enclose the affected area of the body part while extending slightly beyond the affected area to avoid direct contact with the affected area; one or more releasable seals for sealing the enclosure around the body part; a pump in fluid communication with the enclosure for introducing pressurized air into the enclosure; and a pressure gauge for monitoring pressure within the enclosure. A filter device is preferably inserted into a line between the pump and the enclosure to prevent introduction of potentially harmful contaminants (biological or chemical) into the affected area. An air heater/cooler may be incorporated in the pump or placed in-line with the pump to permit control of the air temperature within the enclosure. A computer controller may be provided to receive feedback from the pressure gauge and provide commands to the pump, or to a valve for controlling air flow from the pump, to maintain a selected pressure within the enclosure. In an alternate embodiment, the enclosure may have an openable access port that is substantially air-tight when closed to facilitate access for examination of the affected area without removal of the device.

In another embodiment, the device is configured for alternating pressure and vacuum. In the case of a fully flexible enclosure, i.e., a bag, an internal frame is disposed within the bag to prevent contact between the inner walls of the bag and the affected area when the bag is evacuated. The internal frame may be formed from stainless steel, titanium, plastic, or other polymer that is sufficiently rigid to support the bag and is capable of being safely sterilized to avoid contamination of the affected area.

In the preferred embodiment, the enclosure is a bag formed from pliable plastic or polymer, such as HDPE. Generally, the bag must be capable of retaining air at the desired pressure without significant leakage, i.e., non-porous, and substantially air-tight, including any seams and closures. The bag may be made of a translucent or opaque material, but is preferably transparent, or has a transparent window to permit visual inspection of the affected area without removing the device. The bag material should have minimal elasticity so that it resists expansion once its capacity is achieved. Rubber (natural or synthetic), latex or similar elastic materials may be used in conjunction with some form of limiting structure, such as nylon netting or ribs, or a Neoprene® (polychloroprene) sleeve, to prevent continued expansion of the bag when it is pressurized.

Access to the affected area may be provided by incorporating an optional airtight zipper in the side of the bag. Such zippers are commercially available. Generally, the zipper will run parallel to the length of the body part that is being treated. Other types of airtight closure devices may be used as long as ready access to the body part under treatment is maintained.

The device and method of the present invention are applicable to any body part around which air compression or vacuum can be applied. Since the device is essentially a large balloon surrounding the body part, it can be made large enough to accommodate any hardware surrounding the body part. The pressure in the bag is then inflated with a temperature-controlled air-pump to a pressure between 0 and mean arterial pressure (approximately 80 mmHg). It has been determined that an increased pressure (above atmospheric pressure) of 40 mmHg is the preferred pressure to maximally increase blood flow, however, because different subjects experience maximal increases at different pressures, a personalized approach to compression therapy is appropriate.

Potential applications of the inventive device and method include: healing diabetic foot ulcers; healing venous stasis ulcers, stasis dermatitis; decreasing lower extremity edema; healing fractures, non-unions, and infected non-unions; healing osteomyelitis; healing infected hardware in the extremity; increasing drug delivery to an extremity; healing after plastic surgery, free flap, muscle transposition, and other post-surgical healing; other wound healing in the extremity not specified above; and increasing post-exercise hyperemia for athletic training, rehabilitation, or physical therapy.

In another application of the present invention, preferential delivery of drugs can be effected by increasing blood flow to the area to be treated after the drugs have been introduced orally, by intramuscular injection or IV, or transdermally. In addition, negative pressure on an affected area could be used to remove metabolic end-products, without directly contacting the wound or affected area.

### BRIEF DESCRIPTION OF THE FIGURES

FIG. 1a is a diagrammatic view of a first embodiment of the invention with an optional internal frame structure and a single enclosure seal in contact with the limb; FIG. 1b is a diagrammatic view of an alternate embodiment of the enclosure of FIG. 1a with a seal at each end of the enclosure.

FIG. 2 is a plot of peak systolic velocity of the popliteal artery under varying pressures.

FIG. 3 is a graph of photoplethysmography measurement of tibialis anterior muscle microcirculation.

FIG. 4 is a graph of photoplethysmography measurement of skin blood flow over the anterior tibialis muscle.

FIG. 5 is a graph of photoplethysmography measurement of muscle blood volume of the anterior tibialis muscle.

FIG. 6 is a graph of photoplethysmography measurement of skin blood volume over the anterior tibialis muscle.

### DETAILED DESCRIPTION OF THE PREFERRED EMBODIMENTS

The inventive device comprises a substantially airtight enclosure that is dimensioned to enclose the affected area of the body part plus a margin that extends slightly beyond the affected area to avoid direct contact with, or mechanical pressure against, the affected area when the enclosure is sealed. One or more releasable and resealable, substantially airtight seals are used for sealing the enclosure around the body part at a short distance from the affected area. For purposes of the present invention, a “substantially airtight seal” means that when the enclosure is inflated, the pressurized air can be retained long enough to provide the desired compression over the desired period of time. The enclosure need not be perfectly airtight, particularly if the source of pressurized air, i.e., the pump is activated continuously.

A port is formed in the enclosure for connection to a pump. An air pump is connected to the enclosure via appropriate tubing for introducing air into the enclosure. The pump may include a temperature control device, or a separate temperature control device may be placed either upstream or downstream from the pump, to control the temperature of the air delivered to the enclosure. A pressure gauge is located downstream from the pump, preferably at or near the port for monitoring pressure within the enclosure. A filter device is preferably inserted into the line between the pump and the enclosure to prevent introduction of potentially harmful contaminants (biological or chemical) into the affected area. A computer controller or programmable logic controller (PLC) may be included to receive feedback from the pressure gauge and provide commands to the pump, or to a valve for controlling air flow from the pump, to maintain a selected pressure within the enclosure. The enclosure may have an openable access port that is substantially airtight when closed to facilitate access for examination of the affected area without removal of the device.

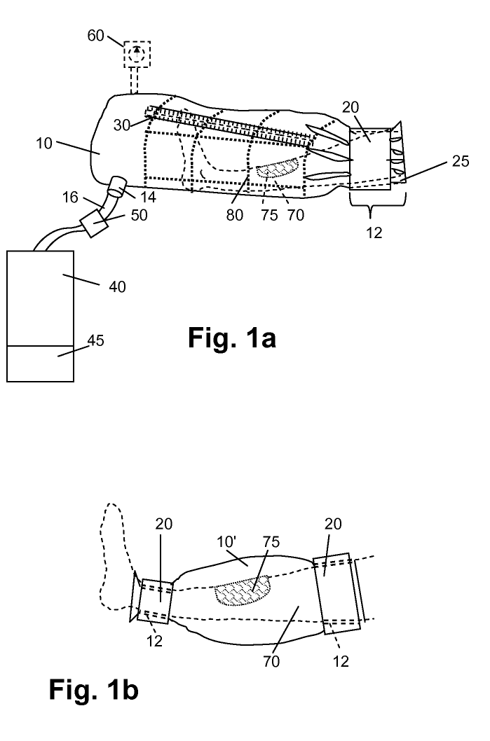

In the exemplary embodiment illustrated in FIG. 1a, bag 10 forms an enclosure that is dimensioned to enclose a leg, an arm, or any part of the body 70, including any supporting structures that might be attached the body, such as an external orthopedic fixator or “halo” device. Bag 10 is preferably formed from a non-porous pliable plastic or polymer sheet, such as an extruded film formed from HDPE (high density polyethylene), LDPE (low density polyethylene), LLDPE (linear low density polyethylene), or other appropriate polymer having a thickness in the range of 0.006-0.15 mm. Alternatively, rubber (natural or synthetic), latex or similar elastic materials may be used in conjunction with some form of limiting structure, such as nylon netting or ribs, or a Neoprene® (polychloroprene) sleeve, to prevent continued expansion of the bag when it is pressurized. Neoprene® alone may also be used as long at it is sufficiently non-porous to remain substantially airtight, e.g., with a plastic coating on one surface of the material. Any seams used to form the bag should be sealed to be substantially airtight.

In the preferred embodiment, the bag is formed from a transparent material, although a translucent, or opaque material may be used. In the case of translucent or opaque material, it may be desirable to incorporate a transparent window in the bag to permit visual inspection of the affected area during treatment. Bag 10 will have at least one open end, as shown in FIG. 1a, or two, as shown in FIG. 1b, to permit location of the affected area within the enclosure. The shape of the bag is not critical since all that is required is that it enclose the area to be treated, plus provide a sufficient margin 12, or selvage, that extends beyond the affected area(s) 75 so that it can be placed in contact with the skin a short distance from, i.e., bordering, but not contacting the area to be treated, for creating a substantially airtight seal. A port 14 is formed in the bag at a location that will not interfere with or contact the body part to permit connection to pump 40. The seal between port 14 and bag 10 should be airtight and sufficiently reinforced to allow repeated attachment and removal of air tubing 16 without damage. A quick release air line coupling may be desirable. Appropriate connectors are commercially available from a wide variety of sources.

After insertion of the body part into the enclosure, the open end 25 is sealed around the body part 70 bordering, and at a sufficient distance from, the area to be treated to avoid contact with the affected area 75. Seal means 12 tightly and uniformly compresses the margin 12 against the skin to create a substantially airtight seal. In the exemplary embodiment, a band having the same or similar construction to a blood pressure cuff is used. The border area over which the cuff is placed should be wide enough to avoid contacting any portion of the area(s) to be treated. In this case, the quality of the seal may be enhanced by slightly inflating the cuff while taking care to avoid restriction of circulation to the affected area. The seal 20 may also be created with Velcro®, elastic, rubber surgical hose, a tourniquet, adhesive tape, or any other means that will secure the bag 10 to the body part 70 to create a substantially airtight seal without occluding blood flow to the area to be treated. When the seal 20 is an inflatable cuff, the pressure source for the cuff may be in electrical communication with a computer controller to read and control the pressure in the cuff.

In the alternate embodiment illustrated in FIG. 1b, a seal 20 is applied over the margins at each open end of the bag 10′ that fits over the body part 70 like a sleeve, allowing a portion of the body part to extend outside of the enclosure.

The seal means 20 need not produce a fully airtight seal, however, if the seal 20 is not substantially airtight, the pump 40 may need to operate continuously to maintain pressure at the desired level.

Pump 40, which can be an air pump or vacuum pump or a combination of both, is connected via tubing 16 to the port in bag 10. The pump 40 may contain an air heating/cooling unit 45 to heat or chill the air that is being used to pressurize the bag 10. The temperature controller 45 may be a separate unit or may be integrated into the pump 40. Pump 40 may pull air from the surrounding environment to pressurize the enclosure, or it may be attached to a purified gas source for introduction of a specified gas, such as oxygen or nitrogen, into the enclosure. If pump 40 is capable of vacuum operation, it may be used to reduce the pressure within bag 10 to less than atmospheric pressure.

A filter 50 is incorporated into the assembly to purify the input air to prevent the introduction of biological and chemical contaminants into the enclosure. As illustrated in FIG. 1a, the filter 50 may be positioned between the pump 40 and the bag 10, however, it may be desirable to locate the filter (or place an additional filter) upstream from the pump to further purify the air that will be pumped into the enclosure. Filter 50 may also be used to remove metabolic end-products when a vacuum is applied to the enclosure.

A pressure gauge 60 is attached to the bag 10 for monitoring the pressure within the bag 10. The pressure gauge 60 may have an analog readout or a digital readout. A computer controller (not shown) may be incorporated into a treatment system according to the present invention to receive a signal generated by the pressure gauge as feedback for control of the pump operation. An optional thermocouple (not shown) may be placed within or in contact with the bag 10, or within tubing 16 or filter 50, to monitor the air temperature and provide feedback to the computer controller for control of the heating/cooling unit.

The computer controller may be programmed for constant pressure, pulsed pressure, constant vacuum, pulsed vacuum or any desired combination. The computer controller may be an integral part of the pump unit or a stand alone component.

To inspect the area that is being treated, an optional airtight zipper 30 may be integrated into the bag 10. This allows for the inspection of the affected area without requiring removal of the body part from the bag 10. Generally, the zipper will run parallel to the length of the body part that is being treated, as shown, however, diagonal or transverse openings may also be used. The zipper 30 may be only a few centimeters in length or run almost the entire length of the bag 10. Access to the affected area may be provided by incorporating an airtight zipper in the side of the bag 10. Such zippers are commercially available and have been used, for example, for hermetic seals for storage containers, and Gamow bags for treatment of high altitude sickness. (See, e.g., Gamow, et al., U.S. Pat. No. 4,974,829, which is incorporated herein by reference.) Other types of airtight closure devices may be used as long as ready access to the body part under treatment is maintained.

When a vacuum pressure is used, an optional frame 80 may be inserted into the bag 10 and around the body part to prevent contact between the inner surface of the bag 10 and the area under treatment. The frame 80 may be made of a rigid plastic, stainless steel, or any other suitable material that can be appropriately sterilized. The frame 80 may be square or circular in shape and may include a shelf or cradle for supporting the body part.

In an alternate embodiment, the enclosure may be a combination of a box formed from a rigid material and a pliable sealing sleeve that extends from an opening in the box to enclose an area of the body beyond the affected area. The rigid material may be acrylic, such as Plexiglass®, polycarbonate (Lexan®), or sheet metal, constructed to be substantially air-tight and sufficiently rigid to resist expansion during increased pressure operation and resist contraction or collapse during vacuum operation. The pliable sealing sleeve may be the same type of plastic sheeting described above for construction of the bag, e.g., HDPE, LDPE or LLDPE, which is attached to the opening of the box to produce a substantially airtight seal when the seal means is applied.

Typical pressures within the enclosure can range from −150 mmHg to +200 mmHg (compared to atmospheric pressure), when both compression and vacuum are used. Otherwise, the compression pressure will range from atmospheric pressure to 200 mmHg. In the preferred embodiment, the pressure in the bag is maintained at about 40 mmHg above ambient pressure. The compression may be continuous at a fixed pressure, ramped up or down, or a cyclic pressure alternating between low (or negative) pressure and high pressure for pre-determined periods may be used as appropriate for the nature of the treatment. The amount of time necessary to achieve maximal blood flows typically occurs within several seconds and lasts at least one hour.

A significant advantage of the present invention is its ability to apply a uniform pressure across the full thickness of the body part thus increasing microcirculatory flow within skin, muscle and deep bone, as shown by the following example.

### Example 1

The leg of a volunteer was inserted into a bag, sealed using a conventional blood pressure cuff, and pressurized to 40 mmHg. The bag was formed from HDPE sheeting, sealed to be substantially airtight. (A Neoprene®sleeve covered by plastic sheeting was also tried and was found easier to apply.) Air tubing was connected between a port formed in the bag and an air pump. The bag was not in contact with the skin. Baseline blood flows were measured using photoplethysmography and Doppler ultrasound. Once the desired pressure level was attained, periodic measurements were taken after which pressurization was terminated and the bag allowed to deflate.

The device was found to increase the large artery inflow to the leg—the popliteal artery peak systolic velocity is increased by about 140%, compared to about 20-25% with existing devices. Pressure treatment increased microvascular skin blood flow in the leg; increased microvascular muscle blood flow in the leg; and modulated extremity blood volume.

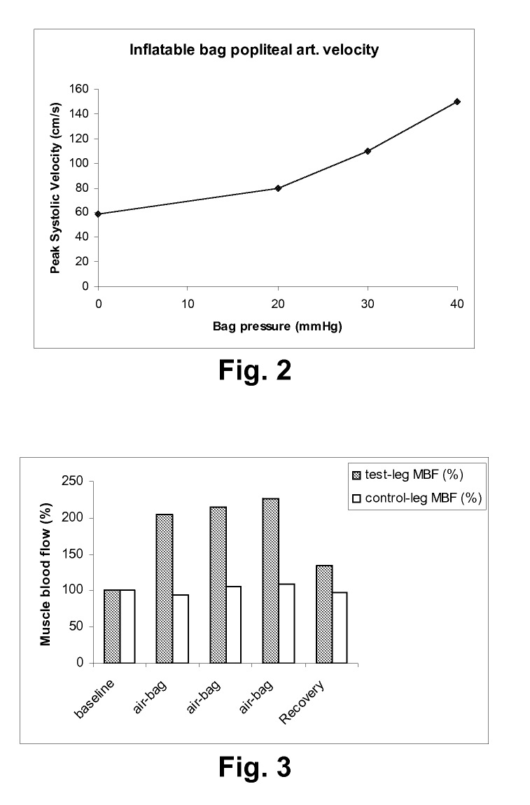

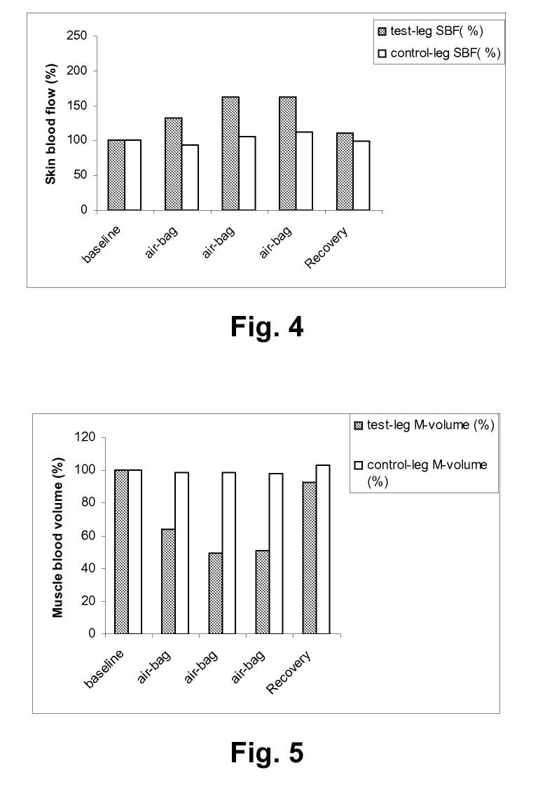

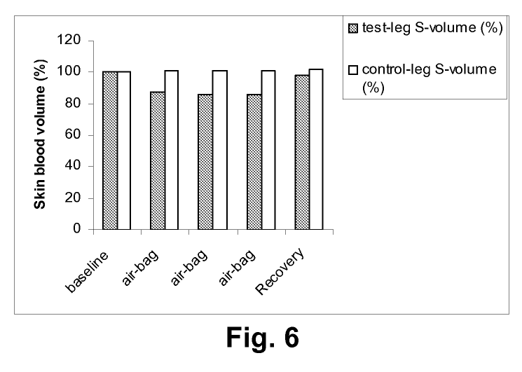

Actual results are shown in FIGS. 2-6. FIG. 2 is a plot of peak systolic velocity of the popliteal artery under varying pressures. Peak systolic velocity increases more than two-fold with pressurization of the bag to 40 mmHg. FIG. 3 is a graph of photoplethysmography measurement of tibialis anterior muscle microcirculation, again showing two-fold or better increases with exposure to compression at 40 mmHg. FIG. 4 is a graph of photoplethysmography measurement of skin blood flow over the anterior tibialis muscle demonstrates more than 1.5-fold increases with bag pressures of 40 mmHg. FIG. 5 is a graph of photoplethysmography measurement of muscle blood volume of the anterior tibialis muscle, which shows a 50% decrease in muscle blood volume (compliant low pressure venous vessels) with bag pressures of 40 mmHg, indicating improved venous blood return to the heart. FIG. 6 is a graph of photoplethysmography measurement of skin blood volume over the anterior tibialis muscle showing approximately 15% decrease with bag pressures of 40 mmHg.

Potential applications of the inventive device and method include: healing diabetic foot ulcers; healing venous stasis ulcers and stasis dermatitis; decreasing lower extremity edema; healing fractures, non-unions, and infected non-unions; healing osteomyelitis; healing infected hardware in the extremity; increasing drug delivery to an extremity; healing after plastic surgery, free flap, muscle transposition, and other post-surgical healing; other wound healing in the extremity not specified above; and increasing post-exercise hyperemia for athletic training, rehabilitation, or physical therapy.

Blood flow rates determine the maximum amount of drug that can be delivered per minute to specific organs and tissues at a given plasma concentration of drug. Tissues that are well perfused can receive a large quantity of drug, provided the drug can cross between the membranes or other barriers present between the plasma and tissue. In another application of the present invention, preferential delivery of drugs can be effected by increasing blood flow to the area to be treated after the drugs have been introduced orally, by intramuscular injection or IV, or transdermally. The drugs may be administered at any point prior to or during exposure of the treatment area to increased air pressure, or even after completion of the predetermine exposure period but prior to return to normal blood flow conditions. Timing will depend on the rate of distribution, diffusion and absorption of the particular drug. In addition, negative pressure on an affected area could be used to remove metabolic end-products, without direct contact to the wound or affected area.

In the foregoing specification, the invention has been described with reference to specific exemplary embodiments thereof. It will, however, be evident that various modifications and changes may be made thereunto without departing from the broader spirit and scope of the invention as set forth in the appended claims and their full scope of equivalents.

### REFERENCES

### Incorporated Herein by Reference

1. Eze A R, Comerota A J, Cisek P L, Holland B S, Kerr R P, Veeramasuneni R, Comerota A J Jr. Intermittent calf and foot compression increases lower extremity blood flow. Am J Surg. 1996 August; 172(2):130-4; discussion 135.
2. Eze A R, Cisek P L, Holland B S, Comerota A J Jr, Verramasuneni R, Comerota A J. The contributions of arterial and venous volumes to increased cutaneous blood flow during leg compression. Ann Vasc Surg. 1998 March; 12(2):182-6.
3. VanBemmelen P S, Mattos M A, Faught W E, Mansour M A, Barkmeier L D, Hodgson K J, Ramsey D E, Sumner D S. Augmentation of blood flow in limbs with occlusive arterial disease by intermittent calf compression. J Vasc Surg. 1994 June; 19(6): 1052-8.
4. Abu-Own A, Cheatle T, Scurr J H, Coleridge Smith P D. Effects of intermittent pneumatic compression of the foot on the microcirculatory function in arterial disease. Eur J Vasc Surg. 1993 September; 7(5):488-92.
5. Delis K T, Labropoulos N, Nicolaides A N, Glenville B, Stansby G. Effect of intermittent pneumatic foot compression on popliteal artery haemodynamics. Eur J Vasc Endovasc Surg. 2000 March; 19(3):270-7.
6. Morris R J, Woodcock J P. Effects of supine intermittent compression on arterial inflow to the lower limb. Arch Surg. 2002 November; 137(11):1269-73.

7. Agu O, Baker D, Seifalian A M. Effect of graduated compression stockings on limb oxygenation and venous function during exercise in patients with venous insufficiency. Vascular. 2004 January; 12(1):69-76.

8. Morris R J, Woodcock J P. Intermittent venous compression, and the duration of hyperaemia in the common femoral artery. Clin Physiol Funct Imaging. 2004 July; 24(4):237-42.

1. An apparatus for increasing blood flow to a body part, comprising:
an enclosure for retaining pressurized air, the enclosure having dimensions for receiving and enclosing an area of the body part to be treated plus a margin for forming a seal at a location of the body part bordering the area to be treated, wherein the enclosure does not physically contact the area to be treated;
a releasable seal means compressing the margin around the body part for creating a substantially airtight seal between an outer surface of the body part and the margin; and
a pump in fluid communication with the enclosure for introducing pressurized air into the enclosure to increase air pressure within the enclosure;
wherein the air pressure within the enclosure is maintained at one or more predetermined levels for a predetermined period of time to apply air compression against the body part.

2. The apparatus of claim 1, further comprising a pressure gauge for monitoring the air pressure within the enclosure.

3. The apparatus of claim 1, further comprising a filter disposed between the pump unit and the bag for removing biological and/or chemical contaminants from the pressurized air.

4. The apparatus of claim 1, wherein the enclosure comprises a bag formed from a pliable material, wherein when air pressure in introduced, the bag inflates.

5. The apparatus of claim 4, wherein the pliable material is sheeting formed from HDPE, LDPE or LLDPE.

6. The apparatus of claim 4, wherein the pliable material is transparent.

7. The apparatus of claim 4, wherein the pliable material is translucent or opaque and further comprising a transparent window formed in a side of the bag.

8. The apparatus of claim 4, further comprising an airtight zipper disposed within the bag for providing access to the area being treated.

9. The apparatus of claim 1, wherein the predetermined pressure level is within the range of atmospheric pressure to 200 mmHg.

10. The apparatus of claim 1, wherein the pump further comprises a vacuum pump for extracting air from the enclosure and the predetermined pressure level alternates between a low pressure and a high pressure within the range of −150 mmHg and +200 mmHg relative to atmosphere.

11. The apparatus of claim 1, further comprising a temperature control device for heating and/or cooling the pressurized air.

12. The apparatus of claim 10, further comprising a frame to prevent collapse of the enclosure during low pressure operation.

13. The apparatus of claim 1, further comprising a computer controller for receiving data corresponding to pressure within the enclosure and controlling the pump to maintain the pressure at the predetermined pressure for the predetermined period of time.

14. A method for increasing blood flow to a body part of an individual under treatment, comprising:
inserting the body part with a treatment area into a sealable enclosure;
releasably sealing the enclosure around the body part at a location of the body part bordering the treatment area so that a substantially airtight seal is created between the surface of the body part and the enclosure, wherein the enclosure does not physically contact the treatment area;
pumping air into the enclosure to increase pressure within the enclosure to a predetermined pressure within the range of 0 to +200 mmHg above atmospheric pressure; and
maintaining increased pressure within the enclosure for a predetermined period of time, wherein air pressure within the enclosure uniformly compresses the body part.

15. The method of claim 14, wherein the enclosure is a bag formed from a pliable sheet of HDPE, LDPE or LLPDE.

16. The method of claim 14, further comprising monitoring the air pressure within the enclosure with a pressure gauge.

17. The method of claim 18, wherein a filter is disposed between the pump unit and the bag for removing biological and/or chemical contaminants from the pressurized air.

18. The method of claim 14, further comprising evacuating air from the enclosure for creating a reduced pressure within the range of −150 mmHg to atmospheric pressure, and alternating evacuation of the air with pumping of air into the enclosure.

19. The method of claim 14, further comprising using a temperature control device for heating and/or cooling the pressurized air.

20. The method of claim 18, further comprising inserting a frame into the enclosure to prevent collapse of the enclosure during reduced pressure operation.

21. The method of claim 14, further comprising using a computer controller for receiving data corresponding to pressure within the enclosure and controlling the pump to maintain the pressure at the predetermined pressure for the predetermined period of time.

22. The method of claim 14, wherein the treatment area is skin over the body part.

23. The method of claim 14, wherein the treatment area is muscle tissue or bone within the body part.

24. The method of claim 14, further comprising administering a drug to the individual under treatment, wherein the increased blood flow causes preferential delivery of the drug to the treatment area.
